(19) United States
(12) Patent Application Publication
(10) Pub. No.: US 2020/0123208 A1
(43) Pub. Date: Apr. 23, 2020

(54) PEPTIDES AND NEMATICIDAL COMPOSITIONS

(71) Applicant: The Queen's University of Belfast, Belfast, Antrim (GB)

(72) Inventors: Neil D. Warnock, Belfast, Antrim (GB); Aaron G. Maule, Belfast, Antrim (GB); Johnathan J. Dalzell, Belfast, Antrim (GB)

(21) Appl. No.: 16/483,245

(22) PCT Filed: Feb. 2, 2018
(86) PCT No.: PCT/EP2018/052709

§ 371 Date: Aug. 2, 2019

(30) Foreign Application Priority Data
Feb. 2, 2017 (GB) 1701750.0

(51) Int. Cl. C07K 14/32; C07K 14/405; C07K 11/00; C07K 7/08; C07K 7/06; A01N 37/46

(52) U.S. Cl. CPC C07K 14/32; C07K 14/405; A01N 37/46; C07K 7/08; C07K 7/06; C07K 11/00

(57) ABSTRACT

Peptides, nematicidal compositions, transgenic microorganisms for expressing the peptides and methods of treating plant parasitic nematodes, the method comprising providing either a peptide or a nematicidal composition or a transgenic microorganism for expressing the peptide on or adjacent the plant parasitic nematodes.

### RELATED APPLICATIONS

This application is a national stage application, filed under 35 U.S.C. § 371, of International Application No. PCT/EP2018/052709 filed Feb. 2, 2018, which claims priority to GB Patent Application No. 1701750.0 filed Feb. 2, 2017, which applications are incorporated herein by reference.

This invention relates to nematode neuropeptides as transgenic nematicides.

Plant parasitic nematodes (PPNs) seriously threaten global food security. Conventionally an integrated approach to PPN management has relied heavily on carbamate, organophosphate and fumigant nematicides which are now being withdrawn over environmental health and safety concerns. This progressive withdrawal has left a significant shortcoming in our ability to manage these economically important parasites, and highlights the need for novel and robust control methods. Nematodes can assimilate exogenous peptides through retrograde transport along the chemosensory amphid neurons. Peptides can accumulate within cells of the central nerve ring and can elicit physiological effects when released to interact with receptors on adjoining cells. We have profiled bioactive neuropeptides from the neuropeptide-like protein (NLP) family of PPNs as novel nematicides, and have identified numerous discrete NLPs that negatively impact chemosensation, host invasion and stylet thrusting of the root knot nematode Meloidogyne incognita, and the potato cyst nematode Globodera pallida.

Transgenic secretion of these peptides from the rhizobacterium, Bacillus subtilis, and the terrestrial microalgae Chlamydomonas reinhardtii reduce tomato infection levels by up to 90% when compared with controls.

These data pave the way for the exploitation of nematode neuropeptides as a novel class of plant protective nematicide, using novel non-food transgenic delivery systems which could be deployed on farmer-preferred cultivars.

Plant parasitic nematodes (PPN) reduce crop plant yield globally, undermining food security. Many of the chemicals used to kill these parasites are non-specific and highly toxic, and are being phased out of general use through governmental and EU regulation. The withdrawal of these chemicals is beneficial to the environment, but limits our ability to protect crops from infection. Efforts must now focus on developing environmentally safe PPN controls. PPNs can absorb various molecules directly from the environment into their nervous system, including peptides and proteins. Here we profiled the feasibility of using PPN neuropeptides, small signalling molecules, to interfere with normal PPN behaviour. We exposed PPNs to a variety of neuropeptides, and found that they could interfere with behaviours that are important to host-finding and invasion. We then developed soil-dwelling microbes that could generate and secrete these neuropeptides into the soil where the PPN infective juveniles are found. These transgenic microbes can protect host plants from infection, and represent a completely new approach to controlling PPNs in crop plants. Importantly, these neuropeptides appear to have no impact on other beneficial nematodes found in the soil.

Plant parasitic nematodes (PPNs) are responsible for an estimated 12.3% reduction in crop yield each year, which equates to losses of around $US80 billion worldwide [1, 2]. Traditionally PPNs have been controlled through the use of fumigant, carbamate and organophosphate nematicides which are being withdrawn over environmental health and safety concerns, through global and EU level directives [3]. The fumigant methyl bromide was used extensively to control PPN infestations for more than 60 years, however the identification of ozone-depleting characteristics was recognised within the Montreal Protocol which aimed to eliminate methyl bromide use by 2010 [4]. Likewise, dibromochloropropane (DBCP), a highly lipophilic brominated organochlorine was first used as a nematicide in the mid 1950's before animal safety tests in the 1960's demonstrated endocrine disrupting, and carcinogenic properties, alongside an increased incidence of developmental defects following exposure. Later studies further demonstrated strong mutagenic properties, and workers at the Occidental Chemical plant in California, which produced DBCP, displayed significantly higher rates of spermatogenic abnormalities relative to the rest of the population [5]. The carbamate nematicide aldicarb also triggers toxicity in non-target organisms through disruption of cholinergic neurons. Initial withdrawal of use across the USA in 1990 was followed by re-introductions to counteract a serious shortfall in alternative control options in 1995; similar dispensations have been afforded to EC states. The extensive withdrawal of frontline nematicides has left a significant shortfall in our ability to control PPNs.

Transgenic approaches could provide a cost-effective means of PPN control. Much effort has focused on the development of in planta RNA interference (RNAi) to silence PPN genes necessary for successful parasitism [6-9]. Whilst many such studies have shown promise, concerns surround the persistence of RNAi trigger-expressing traits. It remains to be established if DNA methylation and transcriptional silencing of double stranded (ds)RNA-expressing transgenes is an issue in plants other than Arabidopsis thaliana [10]. Efforts to inhibit PPN nutrient acquisition through transgenic expression of cystatins that inhibit intestinal protease activity have also proven an effective strategy [6]. The utility of peptide resistance traits has also been demonstrated [7], resulting in field level resistance and high target specificity [8]. Indeed, stacking peptide and cystatin resistance traits has proven extremely effective in plantain, triggering a 99% reduction in PPN infection levels at harvest, with a corresponding 86% increase in plantain yield [9].

Peptides have traditionally been viewed as poor drug candidates due to issues surrounding cellular uptake and half-life. However it has long been known that nematodes display an unusual neuronal uptake mechanism which is exploited by amphid dye-filling methods [11]. The amphid neurons assimilate exogenous peptides which subsequently accumulate in cells of the central nerve ring [11], where they can interact with available receptors.

Neuropeptides are highly enriched and conserved amongst nematodes, coordinating crucial aspects of physiology and behaviour [12-21]. The model nematode Caenorhabditis elegans encodes at least 113 neuropeptide genes, producing over 250 mature neuropeptides [16]. It is thought that this neurochemical diversity underpins the wide array of complex behaviours which are found within such neuroanatomically simple animals [16, 22]. Many neuropeptides are known to be expressed within the anterior neurons of nematodes [16, 22-24], and it is likely that their cognate receptors are expressed in these or adjacent cells. The retrograde transport of exogenous peptides suggests that these receptors could be amenable to activation through signalling molecules following their uptake from the external environment. Conceptually, the mining of native neuropeptide complements for novel nematicides is an attractive prospect, based on the a priori assumption of bioactivity. An additional positive quality of neuropeptides is their characteristically high potency when acting on cognate receptors [13, 25-30]. Furthermore, the high degree of phylogenetic sequence conservation suggests that neuropeptides could represent broad-spectrum nematicides as they share significant sequence similarity within and between parasite species [17, 22, 31, 32]. Disrupting PPN behaviour through the dysregulation of native neuropeptide signalling could hinder the development of resistance traits anchored on target receptor mutation. Selective pressure drives the propagation of drug target variants which escape agonism/antagonism, or the development of enhanced efflux mechanisms [33, 34]. Conceptually, the development of resistance to neuropeptides which coordinate crucial aspects of PPN biology would seem less likely.

Nematode neuropeptide complements are organised into three broad groupings: i) the FMRF-amide Like Peptides (FLPs); the INSulin like peptides (INSs); and iii) the Neuropeptide-Like Proteins (NLPs). FLPs represent the most widely studied and best understood family, characterised by a C-terminal RFamide motif, and are known to coordinate motor and sensory function [14, 16, 22]. In particular, C-terminal amidation is necessary for biological function, and so precludes FLPs from most transgenic delivery methods. INSs coordinate and integrate sensory signals with developmental circuits [35] and they share characteristic domain organisation and tertiary structure with vertebrate insulin peptides [16, 36-40]. Specific proteolytic processing requirements suggest that INSs do not represent ideal candidates for transgenic delivery methods. The NLPs represent the least studied grouping of neuropeptides, comprising every neuropeptide that does not conform to the biosynthetic and structural characteristics of FLPs or INSs and encompassing multiple peptide families. Little is known about their function in nematodes, however many NLPs are expressed in anterior neurons and do not appear to require post-translational modifications [20, 24, 40-45], making them more amenable to generation and delivery by transgenic systems than FLPs or INSs. A key gap in assessing the potential of unamidated NLPs as nematicides is the lack of data on their bioactivity in PPNs.

Jarecki et al (Discovery of neuropeptides in the nematode Ascaris suum by database mining and tandem mass spectroscopy. Journal of Proteome Research. 2011. 10, pp 2098-3106): Tables 2 and 3 identify putative peptides, naming them as A suum nlp-1 to nlp-17, and as A suum nlp-18 to 23 and 34 to 46, respectively. Jarecki et al concludes that predicting and identifying the A suum nlps is an “important first step in understanding the vital role neuropeptides play in the nervous system of A suum”.

McVeigh et al (Neuropeptide-like protein diverstity in phylum Nematoda. International Journal for Parasitology. 2008, 38, pp 1493-1503) identifies nematode neuropeptide-like protein (nlp) sequelogs. Table 1 summarises EST-derived nlp sequelogs in phylum Nematoda and Table 2 indicates their distribution. McVeigh et al provides a first study of the nlp diversity of phylum Nematoda and brings the nematode nlp complement to 46 genes.

Nathoo et al (Identification of neuropeptide-like protein gene families in Caenorhabditis elegans and other species. PNAS. 2001, 98, pp 14000-14005) identifies 32 previously uncharacterised C elegans nlp genes. Nathoo et al concludes that further characterisation of the nlp genes is likely to provide a greater understanding of the mechanisms involved in neuropeptide function in development and behaviour.

Husson et al (Discovering neuropeptides in Caenorhabditis elegans by two dimensional liquid chromatography and mass spectrometry. Biochemical and Biophysical Research Communications. 2005. 335, pp 76-86) identified 21 peptides derived from formerly predicted neuropeptide-like protein precursors and 28 predicted FMRFamide-related peptides. Husson et al sequenced 11 novel peptides derived from 9 peptide precursors.

Warnock et al (Nematode neuropeptides as transgenic nematicides. PLoS Pathogens. 2017. 13 (2), pp 1-20 :e1006237. doi: 10.1371/journal.ppat.1006237. eCollection 2017 Feb.) was published on 27 Feb. 2017, being after the priority date of the present Application.

Here we aimed to characterise the NLP complements in silico for two economically important PPNs that display different modes of infection and parasitism, M. incognita and G. pallida. Subsequently we aimed to screen NLPs for their ability to dysregulate the normal behaviour of infective stage juveniles (J2s) when applied exogenously and, simultaneously, to develop and assess novel transgenic delivery methods as next generation plant protection platforms.

### STATEMENTS OF INVENTION

According to the invention, there is provided a peptide comprising, or consisting of:
AA₁-AA₂-AA₃-F-D-AA₆-AA₇-AA₈-AA₉-AA₁₀-AA₁₁-AA₁₂-AA₁₃-AA₁₄-AA₁₅-AA₁₆-AA₁₇;
wherein AA₁ is selected from S, N and A;
wherein AA₂ is selected from S and A;
wherein AA₃ is selected from S, N and A;
wherein AA₆ is selected from S, L, D and A;
wherein AA₇ is selected from F, S and L;
wherein AA₈ is selected from V, T, M, A, F and G;
wherein AA₉ is selected from G, V and T;
wherein AA₁₀ is selected from R, K, P, S, G and N;
wherein AA₁₁ is selected from G and R;
wherein AA₁₂ is selected from F and G;
wherein AA₁₃ is selected from T and F;
wherein AA₁₄ is selected from G and T;
wherein AA₁₅ is selected from M, L, G and F;
wherein AA₁₆ is selected from D and M; and
wherein AA₁₇ is present or absent and, if present, is selected from T and D.

Optionally, the peptide comprises, or consists of:
AA₁-AA₂-AA₃-F-D-AA₆-AA₇-AA₈-AA₉-AA₁₀-G-F-T-G-AA₁₅-D-AA₁₇;
wherein AA₁ is present or absent and, if present, is selected from S or A;
wherein AA₂ is present or absent and, if present, is S and A;
wherein AA₃ is selected from A, S and N;
wherein AA₆ is selected from S, L and A;
wherein AA₇ is selected from F and L;
wherein AA₈ is selected from V, T, M, A and G;
wherein AA₉ is selected from G and T;
wherein AA₁₀ is selected from R, K, P, S and N;
wherein AA₁₅ is selected from M, L and F; and
wherein AA₁₇ is present or absent and, if present, is selected from D and T.

Further optionally, the peptide comprises, or consists of:
AA₁-AA₂-AA₃-F-D-AA₆-AA₇-AA₈-AA₉-AA₁₀-G-F-T-G-AA₁₅-D-AA₁₇;
wherein AA₁ is absent;
wherein AA₂ is present or absent and, if present, is selected from S and A;
wherein AA₃ is selected from A and S;
wherein AA₆ is selected from S, L and A;
wherein AA₇ is selected from F and L;
wherein AA₈ is selected from V, T, M, A and G;
wherein AA₉ is selected from G and T;
wherein AA₁₀ is selected from R, K, P, S and N;
wherein AA₁₅ is selected from M, L and F; and
wherein AA₁₇ is present or absent and, if present, is T.

Still further optionally, the peptide comprises, or consists of:

Gp-NLP-15a
SFDSLTGPGFTGLDT
Gp-NLP-15b
SFDSFTGPGFTGLD
Gp-NLP-15c
SFDSFTGSGFTGLD
Gp-NLP-15f
SFDSFMGPGFTGMD
Gp-NLP-15h
AFDLFTGPGFTGMD
Gp-NLP-15g
AFDSFTGPGFTGMD
Mi-NLP-15a
AFDSFGTPGFTGFD
Mi-NLP-15b
SFDSFTGPGFTGLD
Mi-NLP-15c
SFDSFVGKGFTGMD
Mi-NLP-15d
AFDSFGTPGFTGFD
Mi-NLP-15e
SAFDSFVGRGFTGMD
Mi-NLP-15f
AFDSFAGNGFTGFD
Mi-NLP-15g
NFDAFMGPGFTGLD
Mi-NLP-15h
AAFDSFVGRGFTGMD

Optionally, the peptide comprises, or consists of:

Mi-NLP-15b
SFDSFTGPGFTGLD
Mi-NLP-15e
SAFDSFVGRGFTGMD

According to the invention, there is provided a peptide comprising
AA₁-G-AA₃-AA₄-AA₅-F-AA₇-AA₈-AA₉-AA₁₀-AA₁₁-AA₁₂-AA₁₃-AA₁₄;
wherein AA₁ is selected from G, S or A;
wherein AA₃ is selected from T, A, I and G;
wherein AA₄ is selected from R or Q;
wherein AA₅ is selected from A, T, L, P and Y;
wherein AA₇ is selected from N, R, Y, M, F, Q, L, A and I;
wherein AA₈ is selected from F, D, M, G, R, V, K and E;
wherein AA₉ is selected from F, D, V, G, H, A, P, G, L, E and F;
wherein AA₁₀ is present or absent and, if present, is selected from A, V, Y, D, G, F and E;
wherein AA₁₁ is present or absent and, if present, is selected from P, S, D, L, Y, E, F, G and A;
wherein AA₁₂ is present or absent and, if present, is selected from P, D, E, M, G, T and D;
wherein AA₁₃ is present or absent and, if present, is selected from D, E, A, K, S, P, L, D, G and Q; and
wherein AA₁₄ is present or absent and, if present, selected from E, L, Q, G, P, F, L, A and E.

Optionally, the peptide comprises, or consists of:

Gp-NLP-21a
GGARAFNFFAPPDE
Gp-NLP-21b
GGARAFNFFAPDE
Gp-NLP-21c
GGTRAFNFFVSDALPSSYE
Gp-NLP-21d
SGIQTFRDDYDEKQAGEL
Gp-NLP-21e
AGGRLFRMVDLPDGDDFVPEG
Gp-NLP-21f
GGARPFYGGGYMDGTW
Gp-NLP-21g
AGGRYFMRHFDDSPFAGWMA
Gp-NLP-21h
GGARAFFGDADGPFNSASYWAP
Gp-NLP-21i
GGARAFNGAEETLLNVANLA
Mi-NLP-9a
AGARAFQRPDFDDASYEL
Mi-NLP-9b
GGARTFLVGE
Mi-NLP-9c
GGARAFAKLEE
Mi-NLP-9d
GGARPFYEE
Mi-NLP-9e
GGARPFYGFFGGGEGTW
Mi-NLP-9f
GGGRYFIRPFADQ

Further optionally, the peptide comprises, or consists of:

Mi-NLP-9f
GGGRYFIRPFADQ

According to the invention, there is provided a peptide comprising A-AA₂-D-AA₄-AA₅-AA₆-AA₇-AA₈-AA₉-AA₁₀-AA₁₁-AA₁₂-AA₁₃-AA₁₄-AA₁₅-AA₁₆;
wherein AA₂ is selected from L or F;
wherein AA₄ is selected from I, V, T, R, M and L;
wherein AA₅ is selected from L or M;
wherein AA₆ is selected from E or D;
wherein AA₇ is selected from S, G, V, D or N;
wherein AA₈ is selected from D or S;
wherein AA₉ is selected from D, G, P or F;
wherein AA₁₀ is selected from F or M;
wherein AA₁₁ is selected from G, M, D, F, L, and I;
wherein AA₁₂ is selected from G, S, F or L;
wherein AA₁₃ is present or absent and, if present, is selected from F, L, D, M or G;
wherein AA₁₄ is present or absent and, if present, is selected from E, A, Q or F;
wherein AA₁₅ is present or absent and, if present, is selected from M or D;
wherein AA₁₆ is present or absent and, if present, is T.

Optionally, the peptide comprises, or consists of:

Gp-NLP-14a
ALDILESDDFGGF
Gp-NLP-14b
ALDVMDGDGFGSFE
Gp-NLP-14c
ALDTLEGDDFMGL
Mi-NLP-8a
AFDRLDVSPFDFDAMT
Mi-NLP-8c
AFDRLEDSGFFGL
Mi-NLP-8d
AFDRLDNSFMLL
Mi-NLP-14a
ALDMLEGDDFIGMQ
Mi-NLP-14b
ALDLMEGDGFGGGFD
Mi-NLP-14c
ALDMMEGDDFIGL

Further optionally, the peptide comprises, or consists of:

Mi-NLP-8d
AFDRLDNSFMLL
Mi-NLP-14c
ALDMMEGDDFIGL

According to the invention, there is provided a peptide that comprises, or consists of:

Mi-NLP-18a
FAPRQFAFA
Mi-NLP-18b
GMRNFAFA
Mi-NLP-18c
SFGDYPFGSRTFAFA
Mi-NLP-18e
SSQFGGENSFARFAFA

Optionally, the peptide comprises, or consists of:

Mi-NLP-18a
FAPRQFAFA

According to the invention, there is provided a peptide that comprises, or consists of:

Gp-NLP-8a
FSDDELAAMPLNDLYLSSPYAFGPF
Gp-NLP-8b
SFDRLEESAFFGQ
Gp-NLP-14d
LNELEGDGFMGLD
Gp-NLP-14e
ALDILDGDDFTGFS
Gp-NLP-14f
ALDALEGNSFGF
Gp-NLP-15d
AAFDTDFTNYD
Gp-NLP-15e
FEPFDGYGFNGFE
Mi-NLP-2
SSLASGRIGFRPA
Mi-NLP-8b
FNDDELSSLPFNFEYFPSLDTH
Mi-NLP-18d
AAENFDENNDIN
Mi-NLP-40
MVSWQPV

The invention also provides a nematicidal composition comprising the aforementioned peptide, or a mixture thereof, and a suitable carrier.

The invention also provides an expression vector comprising the aforementioned peptide.

Optionally, a promoter is operably linked to the aforementioned peptide.

The invention provides a transgenic microorganism for expression of the aforementioned peptide, the microorganism comprising the aforementioned vector. The aforementioned peptide can be provided in a plasmid or, alternatively, the transgene can be incorporated directly into the genome of the microorganism.

The invention provides a method of treating plant parasitic nematodes, the method comprising providing either the aforementioned peptide or the aforementioned nematicidal composition or the aforementioned transgenic microorganism on or adjacent the plant parasitic nematodes, optionally in the rhizosphere of the plant.

Optionally, in the aforementioned peptide, the aforementioned nematicidal composition, the aforementioned vector, the aforementioned transgenic microorganism or the aforementioned method, the peptide comprises, or consists of:

15b
SFDSFTGPGFTOLD
15e
SAFDSFVGROFTGMD
9f
GGGRYFIRPFADQ
18a
FAPRQFAFA
14c
ALDMMEGDDFIGL
8d
AFDRLDNSFMLL

or a mixture thereof.

Further optionally, in the aforementioned peptide, the aforementioned nematicidal composition, the aforementioned vector, the aforementioned transgenic microorganism or the aforementioned method, the peptide comprises, or consists of:

15b
SFDSFTGPGFTGLD

### DRAWINGS

In the drawings,

FIG. 1 shows that exogenous neuropeptides disrupt normal Meloidogyne incognita chemotaxis, plant invasion and stylet thrusting. (A) 100 M. incognita infective stage juveniles (J2s) were incubated in selected uNLPs, and subsequently challenged with an agar plate chemosensory assay (plant root exudate attractant/water control). Each assay of 100 nematode juveniles was repeated ten times. (B) Ten tomato seedlings were individually challenged with 500 M. incognita J2s incubated in selected uNLPs. Number of invading M. incognita J2s were normalised against the negative control group, and expressed as a relative percentage. (C) 100 M. incognita J2s were incubated in selected uNLPs and the frequency of stylet thrusting in response to 5 mM serotonin was counted. Data were normalised to control treated groups. Data shown represent the mean±SEM. *, P<0.05; **, P<0.01; ***, P<0.001; ****, P<0.0001 (One-Way ANOVA & Fisher's LSD; Graphpad Prism 6);

FIG. 2 shows that exogenous neuropeptides disrupt normal Globodera pallida chemotaxis, plant invasion and stylet thrusting. (A) 100 G. pallida infective stage juveniles (J2s) were incubated in selected uNLPs, and subsequently challenged with an agar plate chemosensory assay (plant root exudate attractant/water control). Each assay of 100 nematode juveniles was repeated ten times. (B) Ten tomato seedlings were individually challenged with 500 G. pallida J2s incubated in selected uNLPs. Number of invading G. pallida J2s were normalised against the negative control group, and expressed as a relative percentage. (C) 100 G. pallida J2s were incubated in selected uNLPs and the frequency of stylet thrusting in response to 2 mM serotonin was counted. Data were normalised to control treated groups. Data shown represent the mean±SEM. *, P<0.05; **, P<0.01; ***, P<0.001; ****, P<0.0001 (One-Way ANOVA & Fisher's LSD; Graphpad Prism 6);

FIG. 3 shows that Mi-NLP-15b potently inhibits the chemotaxis and infectivity of Meloidogyne incognita. (A) Serial dilutions of Mi-NLP-15b indicate that J2 chemotaxis is inhibited by low picomolar concentrations. (B) Mi-NLP-15b significantly reduced J2 invasion levels at nanomolar concentrations. Data shown represent the mean±SEM. *, P<0.05; **, P<0.01; ***, P<0.001; ****, P<0.0001 (One-Way ANOVA & Fisher's LSD; Graphpad Prism 6);

FIG. 4 shows transgenic microbes secreting uNLPs protect tomato against Meloidogyne incognita and Globodera pallida. (A) Nine independent Chlamydomonas reinhardtii transformants secreting two distinct nematode neuropeptides (Mi-NLP-9f and Mi-NLP-15b) significantly inhibited the ability of M. incognita J2s to infect tomato plants, with up to 90% protection. (B) Bacillus subtilis cultures secreting either Mi-NLP-15b or Mi-NLP-40 also conferred significant protection against M. incognita J2 invasion. (C) C. reinhardtii transformants secreting Gp-NLP-15b (identical to Mi-NLP-15b) significantly inhibited the ability of G. pallida J2s to invade tomato plants. (D) B. subtilis cultures secreting Gp-NLP-15b also protected tomato plants from G. pallida J2 invasion. Data shown represents mean±SEM. *, P<0.05; **, P<0.01; ***, P<0.001 (One-way ANOVA & Fisher's LSD; Graphpad Prism 6); and

FIG. 5 shows Plant parasitic nematode (PPN) unamidated neuropeptide-like proteins (uNLPs) do not alter Caenorhabditis elegans chemotaxis or Steinernema carpocapsae host-finding. Chemotaxis of mixed stage C. elegans towards the attractants sodium acetate (A), pyrazine (B), benzaldehyde (C), and diacetyl (D) are unaffected by exposure to selected PPN uNLPs. (E) Chemotaxis of S. carpocapsae towards the insect host Galleria mellonella is also unaffected by exposure to selected PPN uNLPs. Data shown represent mean ±SEM (One-way ANOVA & Fisher's LSD; Graphpad Prism 6).

Microorganisms in soil affect soil structure and fertility. Soil microorganisms can be classified as bacteria, actinomycetes, fungi, algae and protozoa. Up to 10 billion bacterial cells inhabit each gram of soil in and around plant roots, a region known as the rhizosphere.

Bacteria

Bacteria and Archaea are the smallest organisms in soil apart from viruses. Bacteria and Archaea are prokaryotic. All of the other microorganisms are eukaryotic. A prokaryote has a very simple cell structure with no internal organelles. Bacteria and archaea are the most abundant microorganisms in the soil, and serve many important purposes, including nitrogen fixation. B. subtilis is commonly found in the upper layers of the soil—the density of spores found in soil is about 10⁶ spores per gram.

Fungi

Fungi are abundant in soil, but bacteria are more abundant. Fungi are important in the soil as food sources for other, larger organisms, pathogens, beneficial symbiotic relationships with plants or other organisms and soil health. Fungi can be split into species based primarily on the size, shape and color of their reproductive spores, which are used to reproduce. Most of the environmental factors that influence the growth and distribution of bacteria and actinomycetes also influence fungi. The quality as well as quantity of organic matter in the soil has a direct correlation to the growth of fungi, because most fungi consume organic matter for nutrition. Fungi thrive in acidic environments, while bacteria and actinomycetes cannot survive in acid, which results in an abundance of fungi in acidic areas. Fungi also grows well in dry, arid soils because fungi are aerobic, or dependent on oxygen, and the higher the moisture content in the soil, the less oxygen is present for them.

Algae

Algae can make their own nutrients through photosynthesis. Photosynthesis converts light energy to chemical energy that can be stored as nutrients. For algae to grow, it must be exposed to light because photosynthesis requires light, so algae are typically distributed evenly wherever sunlight and moderate moisture is available. Algae, do not have to be directly exposed to the sun, but can live below the soil surface given uniform temperature and moisture conditions. Algae are also capable of performing nitrogen fixation. Algae can be split up into three main groups: the Cyanophyceae, the Chlorophyceae and the Bacillariaceae. The Cyanophyceae contain chlorophyll that absorbs sunlight and uses that energy to make carbohydrates from carbon dioxide and water and also pigments that make it blue-green to violet in colour. The Chlorophyceae usually only have chlorophyll in it which makes it green, and the Bacillariaceae contain chlorophyll as well as pigments that make the algae brown in colour. Blue-green algae, or Cyanophyceae, are responsible for nitrogen fixation. The amount of nitrogen they fix depends more on physiological and environmental factors rather than the organism's abilities. These factors include intensity of sunlight, concentration of inorganic and organic nitrogen sources and ambient temperature and stability. Chlamydomonas reinhardtii is a single-cell green alga. Chlamydomonas species are widely distributed worldwide in soil and fresh water.

Protozoa

Protozoa are eukaryotic organisms that were some of the first microorganisms to reproduce sexually, a significant evolutionary step from duplication of spores, like those that many other soil microorganisms depend on. Protozoa can be split up into three categories: flagellates, amoebae and ciliates. Flagellates are the smallest members of the protozoa group, and can be divided further based on whether they can participate in photosynthesis. Nonchlorophyll-containing flagellates are not capable of photosynthesis because chlorophyll is the green pigment that absorbs sunlight. These flagellates are found mostly in soil. Flagellates that contain chlorophyll typically occur in aquatic conditions. Flagellates can be distinguished by their flagella, which is their means of movement. Some have several flagella, while other species only have one that resembles a long branch or appendage. Amoebae are larger than flagellates and move in a different way. Amoebae can be distinguished from other protozoa by their slug-like properties and pseudopodia. A pseudopodia or “false foot” is a temporary obtrusion from the body of the amoeba that helps pull it along surfaces for movement or helps to pull in food. The amoeba does not have permanent appendages and the pseudopodium is more of a slime-like consistency than a flagellum. Ciliates are the largest of the protozoa group, and move by means of short, numerous cilia that produce beating movements. Cilia resemble small, short hairs. They can move in different directions to move the organism, giving it more mobility than flagellates or amoebae.

### Materials and Methods

BLAST Identification of PPN uNLPs

The predicted NLP complement of C. elegans [16] was used in a simple BLASTp and tBLASTn analysis of available genomic/transcriptomic sequence data of G. pallida and M. incognita [46, 47]. All returned hits were curated by eye, and NLPs identified as per McVeigh et al. [17].

PPN Maintenance

M. incognita were maintained in tomato plants (cv. Moneymaker) under greenhouse conditions. 8 weeks post infection M. incognita eggs were harvested from the roots by washing away excess soil and by briefly treating cleaned roots in 5% sodium hypochlorite to soften the root tissue and release the eggs. Eggs were cleaned from debris by passage through nested sieves (180 micron, 150 micron and 38 micron) and washed thoroughly with water. Eggs were separated from remaining soil/silt by centrifugation (2000 rcf for 2 minutes) in 100% sucrose solution and collected in a thin layer of spring water (autoclaved and adjusted to pH 7). Eggs were treated in antibiotic/antimycotic solution (Sigma) overnight, placed in a nylon net with a 38 micron pore size, immersed in spring water and maintained in darkness at 23° C., until infective juveniles emerged. Freshly hatched juveniles were used for each assay.

G. pallida were maintained in potato (cv. Cara) at the Agri-Food and Biosciences Institute (AFBI), Belfast. Soil was collected surrounding potato roots, dried for one week and washed through sieves to collect cysts. Cysts were incubated in potato root diffusate in the dark at 17° C. until infective juveniles emerged. Freshly hatched juveniles were used for each assay.

PPN uNLP Screen

Predicted uNLPs from both M. incognita and G. pallida were synthesised by EZBiolab and dissolved into pH adjusted ddH₂O to make a 5 mM stock which was aliquoted and stored at −20° C. J2s of both M. incognita and G. pallida were incubated for 24 hours in 200 μl of each peptide in a 24 well plate (SPL Lifesciences, South Korea) at a defined concentration.

PPN uNLP Screen: Chemosensory Assays

A 60 mm Petri dish was divided into two segments, a positive and a negative side, with a 0.5 cm ‘dead zone’ either side of the centre point. The petri dish was filled with 15 ml of 0.25% w/v agar which was allowed to solidify. 3 ml of 0.25% w/v agar slurry in spring water (pH 7, agitated with a magnetic stirrer for several hours to give a smooth consistency) was added to the petri dish and spread evenly over the surface. Root diffusate (attractant) and water only (control) 0.25% agar plugs were embedded in the agar slurry, either side of the assay arena. Root diffusate was collected from 10 tomato plants, aged 3-6 weeks in 1 litre pots, by pouring 500 ml of ddH₂O through the soil three times. Diffusate from each plant was combined, filter sterilised and stored at 4° C. for a maximum of 1 month. Root diffusate agar plugs were made by melting 1.25% agar in ddH₂O, cooling to 50° C. before mixing with 4 parts of root diffusate. The agar was then allowed to solidify at room temperature. 100 uNLP pre-treated M. incognita or G. pallida J2s were added by pipette to the centre of the plate. J2s which moved out of the ‘dead zone’ after 3 hours were counted and their location (+/−) scored. The distribution of J2s were used to create a chemotaxis index [68] for each plate, which formed one replicate, a total of 10 replicates where completed for each uNLP treatment.

PPN uNLP Screen: Tomato Invasion Assays

Tomato seeds were sterilised with 2.5% NaOCl for 15 minutes, washed 5 times in ddH₂O and germinated on 0.5% Murashige and Skoog plates at 23° C. An agar slurry was prepared by autoclaving 0.55% (w/v) agar (using autoclaved spring water adjusted to pH 7) which was mechanically agitated overnight until it had a smooth consistency. Invasion assays were performed by mixing 500 pre-treated M. incognita or G. pallida J2s with agar slurry and a single tomato seedling (2 days post germination) in a 6 well plate. Assays were left at 23° C. for 24 hours in the case of M. incognita and at 18° C. for 24 hours in the case of G. pallida under a 16 hour light and 8 hour darkness cycle. Seedlings were stained using acid fuschin [69] and the number of nematodes within the roots counted.

PPN uNLP Screen: Stylet Thrusting Assays

Stylet thrusting assays where performed by incubating 100 M. incognita or G. pallida J2s for 15 minute in 5 mM or 2 mM serotonin (Sigma Aldrich, USA), respectively. J2s were placed on a glass slide and stylet thrusts were counted for randomly selected J2s, for 1 minute each. Counting took place for a maximum of 15 minutes. Longer incubations yielded inconsistent results. At least 30 J2s were counted for each neuropeptide treatment.

B. subtilis and C. reinhardtii Plant Protection Assays

B. subtilis were grown overnight in LB media containing ampicillin (100 μg/ml) at 37° C. with shaking, and harvested in the log phase of growth determined by measuring OD₆₀₀ₙₘ. Five ml of culture at 0.5 OD was spun down and the pellet mixed with 3 ml of agar slurry and 500 J2s from either G. pallida or M. incognita. C. reinhardtii clones were grown at 23° C. with shaking, cultures in the log phase were measured at OD₇₅₀ and 5 ml of culture at 0.5 OD was pelleted by centrifugation. C. reinhardtii pellets were mixed with 3 ml of agar slurry and 500 J2s from either G. pallida or M. incognita. Plant invasion assays were performed as described above.

C. elegans Culture and Assays

C. elegans wild-type N2 Bristol strain were obtained from the C. elegans Genomics Center and maintained on a Escherichia coli (strain OP50) lawn on nematode growth medium (NGM) agar plates (3 g/l NaCl, 17 g/l agar, 2.5 g/l peptone, 5 mg/l cholesterol, 25 mM KH₂PO₄ (pH 6.0), 1 mM CaCl₂, 1 mM MgSO₄) at 20° C. [70]. Chemotaxis assays were performed in a 9 cm diameter Petri dish on NGM agar which was split into a positive and negative side with a central ‘dead zone’ of 1.5 cm diameter. 100 mixed-staged C. elegans were washed three times in M9 buffer and soaked in 100 μM PPN uNLP, or M9 vehicle control for 24 hours. 2 μl of 50 mM sodium acetate, 0.5% pyrazine, 0.5% benzaldehyde or 0.5% diacetyl was spotted onto the positive side, 2 μl of ddH₂O was spotted onto the negative side. Pyrazine, benzaldehyde and diacetyl volatile attractants were assayed immediately whereas the water soluble sodium acetate was assayed 18 hours following addition to the plate. Assays were maintained in the dark at 20° C., and counted after 1 hour.

S. carpocapsae Culture and Host-Finding Assay

S. carpocapsae were cultured in Galleria mellonella at 23° C. Infective juveniles (IJs) were collected using a White trap [71] in PBS. Freshly emerged IJs were used for each assay. 100 IJs were incubated for 24 hours in 100 μM of selected uNLPs, and host-finding assays performed as in Morris et al. [45].

Construction of uNLP Expression/Secretion Plasmids

Codon optimised DNA sequences coding for the desired neuropeptide flanked by restriction sites necessary to clone into the C. reinhardtii expression vector pChlamy_3 (Life Technologies, USA) or the B. subtilits expression vector pBE-S (Clontech, USA) were synthesised by GeneArt® Gene Synthesis (Life Technologies, USA).

Transformation of C. reinhardtii

uNLP secretion inserts, and vector pChlamy_3 were digested using Kpnl/Xbal (New England Biolabs, USA), ligated using T4 ligase (New England Biolabs, USA), and cloned into Escherichia coli One Shot® TOP10 chemically competent cells (Life Technologies, USA) following manufacturer's instructions. Ampicillin (Sigma Aldrich, USA) was used to select E. coli containing the pChlamy_3 plasmid, which was subsequently extracted using the High Pure Plasmid Isolation Kit (Roche) and sequenced (Eurofins Genomics, UK) to identify correct clones. C. reinhardtii was transformed by electroporation following manufacturer's instructions (GeneArt® Chlamydomonas Engineering Kit, Life Technologies) and individual colonies grown on TAP-Agar-Hygromycin plates (10 μg/mL) (Sigma Aldrich, USA) at 23° C. Colonies were picked and grown at 23° C. in 100 ml TAP growth media (Invitrogen, USA) with constant orbital agitation. qRT-PCR was performed to identify clones with the highest level of uNLP expression, which were then selected for downstream assays (pChlamy universal FWD: CACTTTCAGCGACAAACGAG, nlp-15b REV: CTACTAGTCGAGGCCGGTA; Mi-nlp-9f REV: GAACGGGCGGATGAAGTAG).

Transformation of B. subtilis

uNLP secretion inserts, and vector pBE-S were digested using Xbal/Mlul (New England Biolabs, USA), ligated using T4 ligase (New England Biolabs, USA), and cloned into E. coli One Shot® TOP10 chemically competent cells (Life Technologies, USA) following manufacturer's instructions. Ampicillin (Sigma Aldrich, USA) was used to select E. coli containing the pBE-S plasmids, which were subsequently extracted using the High Pure Plasmid Isolation Kit (Roche) and sequenced (Eurofins Genomics, UK) to identify correct clones. B. subtilis RIK1285 competent cells (Takara, USA) were transformed according to manufacturer's instructions and grown overnight at 37° C. on kanamycin selective plates (10 μg/mL) (Sigma Aldrich, USA). Individual colonies were picked and grown in LB broth overnight at 37° C. qRT-PCR (pBE-S universal FWD: GGATCAGCTTGTTGTTTGCGT, nlp-15b REV: CCTGGCCCAGTGAAAGAGTC, Mi-nlp-40 REV: TACCGGCTGCCAAGATACCA) was performed to confirm the expression of uNLP secretion cassettes.

Statistical Analysis

Data pertaining to behavioural and invasion assays were assessed by Brown-Forsythe and Bartlett's tests to examine homogeneity of variance between groups. One-way ANOVA was followed by Fisher's Least Significant Difference (LSD) test. All statistical tests were performed using GraphPad Prism 6.

### Results

BLASTp Identification of Predicted NLPs

Pro-peptide sequences of C. elegans NLPs predicted to be unamidated (no C-terminal glycine; uNLPs) were used as queries to conduct a BLASTp analysis of the predicted protein complements of both M. incognita and G. pallida [46, 47]. A total of four nlp genes encoding 25 predicted uNLPs were found within the G. pallida genome, and seven nlp genes encoding 28 predicted uNLPs within the M. incognita genome (Table 1).

TABLE 1
The predicted unamidated NLP complements of Globodera pallida and
Meloidogyne incognita.

| Globodera pallida NLPs* | | Meloidogyne incognita NLPs* | |
|---|---|---|---|
| Gp-NLP-8a | FSDDELAAMPLNDLYLSSPYAFGPF | Mi-NLP-2 | SSLASGRIGFRPA |
| Gp-NLP-8b | SFDRLEESAFFGQ | Mi-NLP-8a | AFDRLDVSPFDFDAMT |
| Gp-NLP-14a | ALDILESDDFGGF | Mi-NLP-8b | FNDDELSSLPFNFEYFPSLDTH |
| Gp-NLP-14b | ALDVMDGDGFGSFE | Mi-NLP-8c | AFDRLEDSGFFGL |
| Gp-NLP-14c | ALDTLEGDDFMGL | Mi-NLP-8d | AFDRLDNSFMLL |
| Gp-NLP-14d | LNELEGDGFMGLD | Mi-NLP-9a | AGARAFQRPDFDDASYEL |
| Gp-NLP-14e | ALDILDGDDFTGFS | Mi-NLP-9b | GGARTFLVGE |
| Gp-NLP-14f | ALDALEGNSFGF | Mi-NLP-9c | GGARAFAKLEE |
| Gp-NLP-15a | SFDSLTGPGFTGLDT | Mi-NLP-9d | GGARPFYEE |
| Gp-NLP-15b | SFDSFTGPGFTGLD | Mi-NLP-9e | GGARPFYGFFGGGEGTW |
| Gp-NLP-15c | SFDSFTGSGFTGLD | Mi-NLP-9f | GGGRYFIRPFADQ |
| Gp-NLP-15d | AAFDTDFTNYD | Mi-NLP-14a | ALDMLEGDDFIGMQ |
| Gp-NLP-15e | FEPFDGYGFNGFE | Mi-NLP-14b | ALDLMEGDGFGGGFD |
| Gp-NLP-15f | SFDSFMGPGFTGMD | Mi-NLP-14c | ALDMMEGDDFIGL |
| Gp-NLP-15g | AFDSFTGPGFTGMD | Mi-NLP-15a | AFDSFGTPGFTGFD |
| Gp-NLP-15h | AFDLFTGPGFTGMD | Mi-NLP-15b | SFDSFTGPGFTGLD |
| Gp-NLP-21a | GGARAFNFFAPPDE | Mi-NLP-15c | SFDSFVGKGFTGMD |
| Gp-NLP-21b | GGARAFNFFAPDE | Mi-NLP-15d | AFDSFGTPGFTGFD |
| Gp-NLP-21c | GGTRAFNFFVSDALPSSYE | Mi-NLP-15e | SAFDSFVGRGFTGMD |
| Gp-NLP-21d | SGIQTFRDDYDEKQAGEL | Mi-NLP-15f | AFDSFAGNGFTGFD |
| Gp-NLP-21e | AGGRLFRMVDLPDGDDFVPEG | Mi-NLP-15g | NFDAFMGPGFTGLD |
| Gp-NLP-21f | GGARPFYGGGYMDGTW | Mi-NLP-15h | AAFDSFVGRGFTGMD |
| Gp-NLP-21g | AGGRYFMRHFDDSPFAGWMA | Mi-NLP-18a | FAPRQFAFA |
| Gp-NLP-21h | GGARAFFGDADGPFNSASYWAP | Mi-NLP-18b | GMRNFAFA |
| Gp-NLP-21i | GGARAFNGAEETLLNVANLA | Mi-NLP-18c | SFGDYPFGSRTFAFA |
| | | Mi-NLP-18d | AAENFDENNDIN |
| | | Mi-NLP-18e | SSQFGGENSFARFAFA |
| | | Mi-NLP-40 | MVSWQPV |

*, single letter annotation of amino acids.

uNLPs Dysregulate Key Behaviours of M. incognita J2s

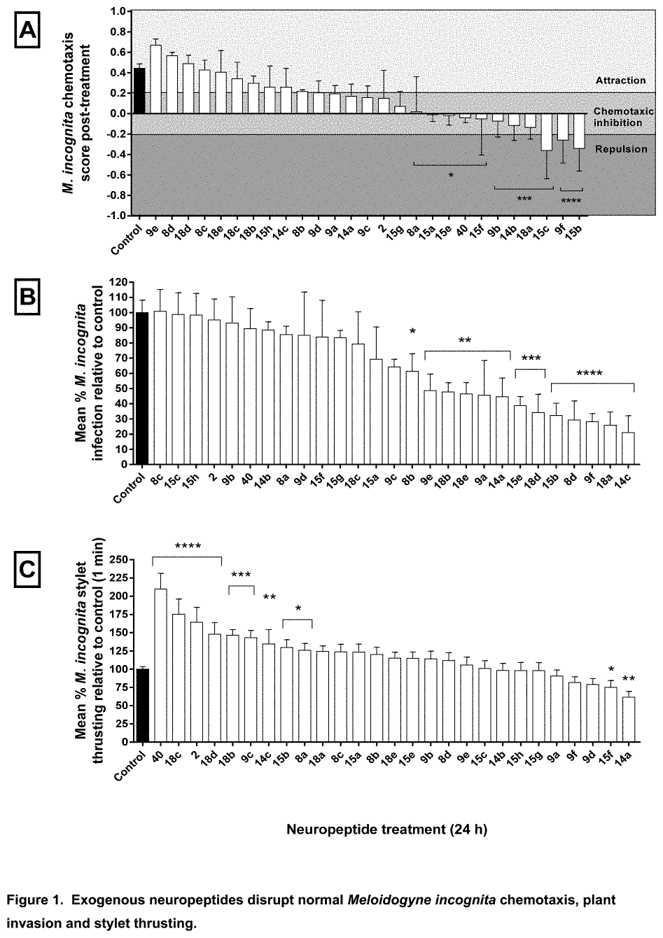

Predicted uNLPs were synthesised and screened against M. incognita and G. pallida J2s for plant protective qualities. Chemotaxis, host-invasion, and stylet thrusting behaviours were assayed following J2 exposure to 100 μM of each uNLP for 24 h. Eleven of 27 tested uNLPs were found to disrupt normal chemotaxis towards root exudate: Mi-NLP-8a (Cl: 0.018+/−0.3438, p=0.0416), Mi-NLP-15a (Cl: −0.01067+/−0.06497, p=0.0299), Mi-NLP-15e (Cl: −0.01767+/−0.09428, p=0.0275), Mi-NLP-40 (Cl: −0.04+/−0.04726, p=0.021), Mi-NLP-15f (Cl: −0.05+/−0.3547, p=0.0185), Mi-NLP-9b (Cl: −0.07417+/−0.154, p=0.001), Mi-NLP-14b (Cl: −0.1155+/−0.1472, p=0.0004), Mi-NLP-18a (Cl: −0.1353+/−0.1129, p=0.006), Mi-NLP-9f (Cl: −0.26+/−0.224, p<0.0001), Mi-NLP-15b (Cl: −0.3408+/−0.2207, p<0.0001), Mi-NLP-15c (Cl: −0.359+/−0.277, p=0.0002) (FIG. 1A).

Likewise, 13 uNLPs were also found to disrupt M. incognita host invasion compared to controls: Mi-NLP-8b (61.38%+/−11.56, p=0.0113), Mi-NLP-9e (48.68%+/−10.87, p=0.0022), Mi-NLP-18b (47.81%+/−6.008, p=0.0019), Mi-NLP-18e (46.49%+/−7.391, p=0.0014), Mi-NLP-9a (45.59%+/−22.97, p=0.0039), Mi-NLP-14a (44.61%+/−12.25, p=0.0033), Mi-NLP-15e (38.71%+/−5.963, p=0.0003), Mi-NLP-18d (34.21%+/−12, p=0.0001), Mi-NLP-15b (32.22%+/−8.122, p<0.0001), Mi-NLP-8d (29.31%+/−12.57, p<0.0001), Mi-NLP-9f (28.22%+/−5.253, p<0.0001), Mi-NLP-18a (25.88%+/−8.695, p<0.0001), Mi-NLP-14c (20.96%+/−11.12, p<0.0001) (FIG. 1B).

Eleven uNLPs were also found to disrupt the rate of serotonergic-induced M. incognita stylet thrusting (positively or negatively) compared with controls: Mi-NLP-40 (210%+/−21.35, p<0.0001), Mi-NLP-18c (175.5%+/−20.72, p<0.0001), Mi-NLP-2 (164.4%+/−20.3, p<0.0001), Mi-NLP-18d (147.7%+/−16.05, p<0.0001), Mi-NLP-18b (146.6%+/−7.609, p=0.0002), Mi-NLP-9c (143.2%+/−9.878, p=0.0005), Mi-NLP-14c (134.7%+/−19.51, p=0.0053), Mi-NLP-15b (129.9%+/−10.59, p=0.0159), Mi-NLP-8a (126.1%+/−9.278, p=0.0359), Mi-NLP-15f (75.18%+/−9.199, p=0.0456), Mi-NLP-14a (61.6%+/−7.86, p=0.002) (FIG. 1C).

uNLPs Dysregulate Key Behaviours of G. pallida J2s

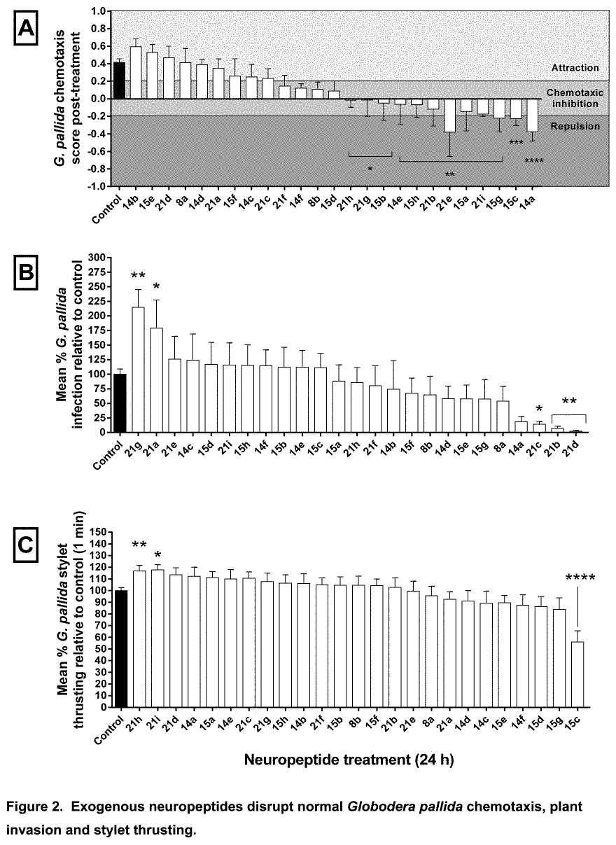

12 of 25 tested uNLPs were found to disrupt chemotaxis of G. pallida J2s towards root exudate: Gp-NLP-21f (Cl: 0.1456+/−0.1232, p=0.0317), Gp-NLP-21g (Cl: −0.01357+/−0.1854, p=0.0248), Gp-NLP-21h (Cl: −0.01564+/−0.08072, p=0.0488), Gp-NLP-15b (Cl: −0.04762+/−0.1983, p=0.0345), Gp-NLP-14e (Cl: −0.0641+/−0.2329, p=0.0026), Gp-NLP-15h (Cl: −0.06784+/−0.1415, p=0.0052), Gp-NLP-21b (Cl: −0.117 +/−0.1936, p=0.0008), Gp-NLP-15a (Cl: −0.1451 +/−0.221, p=0.0035), Gp-NLP-21i (Cl: −0.1733 +/−0.02667, p=0.0074), Gp-NLP-15g (Cl: −0.2208 +/−0.1568, p=0.001), Gp-NLP-15c (Cl: −0.227+/−0.0776, p=0.0002), Gp-NLP-14a (Cl: −0.3765+/−0.1039, p<0.0001), Gp-NLP-21e (Cl: −0.3804+/−0.2762, p<0.0001) (FIG. 2A).

Five uNLPs disrupt G. pallida host invasion relative to controls: Gp-NLP-21d (2.01%+/−1.545, p=0.004), Gp-NLP-21c (14.07%+/−4.655, p=0.0115), Gp-NLP-21a (178.9%+/−48.52, p=0.0201), Gp-NLP-21b (6.897%+/−3.855, p=0.0086), Gp-NLP-21g (214.8%+/−30.31, p=0.0012) (FIG. 2B).

Three uNLPs were also found to modulate serotonergic-induced stylet thrusting of G. pallida J2s relative to controls groups: Gp-NLP-21 i (117.7%+/−4.497, p=0.0302), Gp-NLP-21 h (116.8%+/−4.876, p=0.0046), Gp-NLP-15c (56.07%+/−9.441, p<0.0001) (FIG. 2C).

Mi-NLP-15b Inhibits M. incognita Chemotaxis and Host Invasion with High Potency

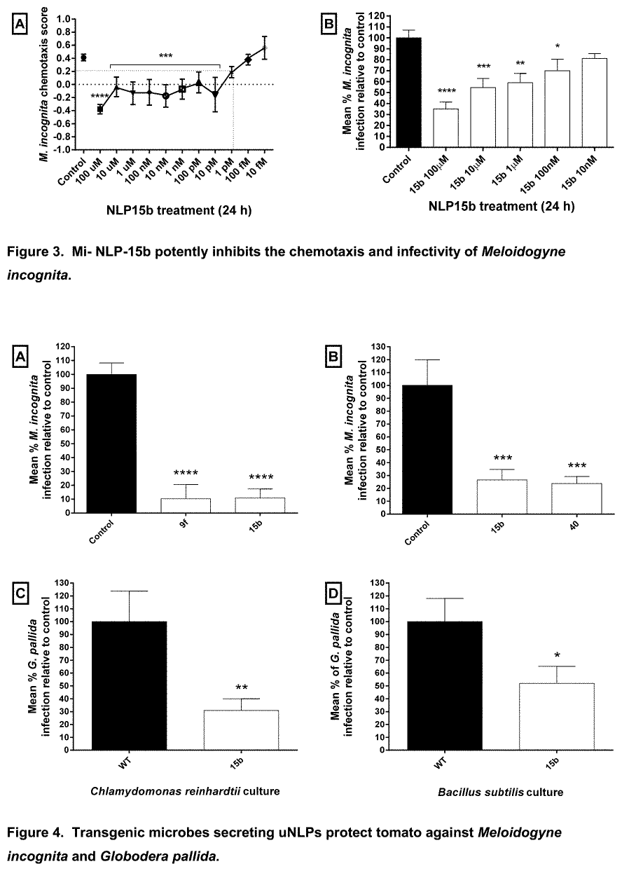

The potency of Mi-NLP-15b-induced disruption of chemotaxis and host invasion was assessed by exposing M. incognita J2s to various concentrations of synthetic Mi-NLP-15b for 24 h. Normal chemotaxis of M. incognita towards root exudate was inhibited across a range of dilutions, indicating high potency: 100 μM, (Cl: −0.3782+/−0.07224, p=0.0031), 10 μM, (Cl: −0.03579+/−0.1504, p=0.0025), 1 μM, (Cl: −0.1344+/−0.1733, p=0.001), 100 nM, (Cl: −0.1195+/−0.1968, p=0.0014), 10 nM, (Cl: −0.1741+/−0.1724, p=0.0055), 1 nM, (Cl: −0.07105+/−0.1534, p=0.0035), 100 pM, (Cl: 0.03117+/−0.1594, p=0.0202), 10 μM, (Cl: −0.1553+/−0.2642, p=0.0037) (FIG. 3A). We found that M. incongita J2 invasion was also inhibited across a range of Mi-NLP-15b concentrations: 100 μM, (35.06%+/−6.407, p<0.0001), 10 μM, (54.62%+/−8.362, p=0.0002), 1 μM, (59.05%+/−8.545, p=0.0036), 100 nM, (69.9%+/−10.66, p=0.0295) (FIG. 3B).

Transgenic Microbes Secreting uNLPs Protect Plants from PPN Invasion

Innoculation of C. reinhardtii cultures secreting selected uNLPs into the tomato invasion assay arena inhibited M. incognita invasion relative to untransformed C. rehinhardtii: Mi-NLP-9f (10.32%+/−10.32, p<0.0001), Mi-NLP-15b (10.82%+/−6.574, p<0.0001) (FIG. 4A). Likewise, innoculation of B. subtilis cultures secreting selected uNLPs, significantly inhibited M. incognita invasion: Mi-NLP-15b (26.63%+/−8.12, p=0.0003), Mi-NLP-40 (23.72%+/−5.448, p=0.0002) (FIG. 4B). C. reinhardtii expressing Gp-NLP-15b also inhibited G. pallida invasion relative to controls (30.95%+/−9.021, p=0.0042) (FIG. 4C). Similarly, innoculation with B. subtilis secreting Gp-NLP-15b inhibited G. pallida invasion relative to control groups (51.98%+/−13.29), p=0.0203 (FIG. 4D).

PPN uNLPs do not Alter Behaviours of Non-Target Nematodes

BLAST was used to identify NLP-15b homologues across available expressed sequence tags (ESTs) or genomes of PPNs and non-target nematode species. PPNs with diverse life history traits share high levels of NLP-15b sequence similarity, however sequence similarity is reduced in non-target nematode species (Table 2).

TABLE 2
Sequence alignment of NLP-15b in selected
parasitic nematode species and the free
living nematode C. elegans.

| Nematode Species | NLP-15b sequence* |
|---|---|
| Meloidogyne incognita | SFDSFTGPGFTGLD |
| Meloidogyne javanica | SFDSFTGPGFTGLD |
| Meloidogyne hapla | SFDSFTGPGFTGLD |
| Meloidogyne chit woodi | SFDSFMGPGFTGLD |
| Globodera paffida | SFDSFTGPGFTGLD |
| Globodera rostochiensis | SFDSFTGPGFTGLD |
| Heterodera glycines | SFDSFTGPGFTGLD |
| Pratylenchus penetrans | SFDSFMGPGFTGLD |
| Radopholus similis | SFDSFMGPGLTGLD |
| Steinemema carpocapsae | AFDSFMGSGFTGMD |
| Pristionchus pacifius | SFDTFGGVRFSPLE |
| Caenorhabditis elegans | AFDSLAGSGFGAFN |

*, single letter annotation of amino acids.

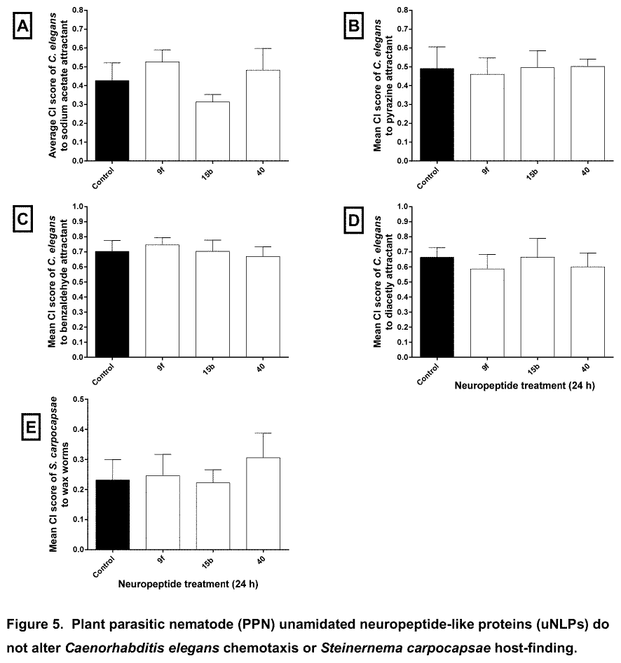

Incubation of mixed-stage C. elegans in selected PPN uNLPs (100 μM, 24 h) had no statistically significant impact on chemotaxis towards: (i) sodium acetate. Mi-NLP-9f (Cl: 0.5261+/−0.064, p=0.4164), Mi/Gp-NLP-15b (Cl: 0.3142+/−0.039, p=0.3578) or Mi-NLP-40 (Cl: 0.4820+/−0.115, p=0.6485) relative to control groups (Cl: 0.4269+/−0.094), (FIG. 5A); (ii) pyrazine. Mi-NLP-9f (Cl: 0.4599+/−0.087, p=0.8094), Mi/Gp-NLP-15b (Cl: 0.4959+/−0.089, p=0.9648) or Mi-NLP-40 (Cl: 0.5018+/−0.039, p=0.9282) relative to controls (Cl: 0.4904+/−0.116) (FIG. 5B); (iii) benzaldehyde. Mi-NLP-9f (Cl: 0.7463+/−0.047, p=0.6416), Mi/Gp-NLP-15b (Cl: 0.7032+/−0.075, p=0.9952) or Mi-NLP-40 (Cl: 0.6686+/−0.065, p=0.7172) relative to controls (Cl: 0.7026+/−0.072) (FIG. 5C); (iv) diacetyl. Mi-NLP-9f (Cl: 0.6+/−0.092, p=0.5739), Mi/Gp-NLP-15b (Cl: 0.6640+/−0.126, p=0.9987) or Mi-NLP-40 (Cl: 0.5858+/−0.097, p=0.6454) relative to controls (Cl: 0.6638+/−0.064) (FIG. 5D). Exposure of S. carpocapsae infective juveniles (IJs) to selected PPN uNLPs also had no statistically significant impact on insect host-finding: control (Cl: 0.2315+/−0.068); Mi-NLP-9f (Cl: 0.2462+/−0.070, p=0.8784); Mi/Gp-NLP-15b (Cl: 0.2225+/−0.043, p=0.9249); or, Mi-NLP-40 (Cl: 0.3057+/−0.082, p=0.4422) (FIG. 5E).

Discussion

We have identified seven nlp genes which putatively encode 27 mature unamidated peptides in the root knot nematode, M. incognita. Likewise, four nlp genes predicted to encode 24 mature unamidated peptides were identified in the potato cyst nematode, G. pallida (Table 1). Several predicted unamidated NLPs share high levels of amino acid sequence similarity between M. incognita and G. pallida, with one predicted peptide, designated NLP-15b, perfectly conserved between the two. Indeed, NLP-15b is highly conserved at the sequence level across PPN species with diverse life history traits; less sequence similarity is observed between NLP-15b from PPNs and non-target species such as S. carpocapsae, C. elegans or P. pacificus for example (see Table 2).

Selected M. incognita and G. pallida peptides had a negative impact on PPN chemosensation and host-finding behaviours, but not on chemosensory or host-finding behaviours of mixed stage C. elegans or S. carpocapsae infective juveniles (FIG. 1, 2, 5). This may be due to NLP sequence dissimilarity, or to different peptide uptake efficiencies between species. The attractants used to assay C. elegans chemotaxis operate via distinct neuroanatomical and biochemical pathways; sodium acetate is detected by the ASE neurons, benzaldehyde by the AWC neurons and prazine and diacetyl are both detected by the AWA neuron [48, 49]. Off-target NLP impacts were also assessed as a factor of host-finding ability in S. carpocapsae which will involve numerous neuroanatomical and biochemical pathways. Whilst these data on C. elegans and S. carpocapsae are far from exhaustive, they suggest that neuropeptide treatments which produce strong disruptive effects on the behaviours of M. incognita and G. pallida may be specific to PPNs.

Whilst it is tempting to extrapolate something on native NLP functionality from these data, we do not know if the aberrant phenotypes observed are due to interactions between tested NLPs and their cognate receptors. However, we do observe that exogenous NLPs can interact with endogenous neurophysiological circuits, interfering with host-finding, invasion and serotonergic stylet-thrusting behaviours of both M. incognita and G. pallida juveniles (FIG. 1, 2). This supports our initial hypothesis that nematode neuropeptides represent a valuable repository of nematicide candidates, which may elicit broad-spectrum activities against PPN species, but not off-target nematode species. Serial dilution of Mi-NLP-15b inhibited M. incognita chemosensation at concentrations as low as 10 pM, demonstrating high uNLP potency, which is a known characteristic of interactions between nematode neuropeptides and their cognate receptors [13, 25-30, 50, 51] (FIG. 3). While the potency of this peptide would support the specificty of the associated phenotypic impact, we advise some caution when interpreting these data as indicative of NLP function within either M. incognita or G. pallida J2s due to the potential for peptide interaction with other, non-cognate receptors.

In order to further assess the efficacy of exogenous NLPs as nematicides, we developed two transgenic synthesis and delivery systems which could be deployed in field, potentially through seed treatments or soil amendments. Gram positive Bacillus spp. are a major component of rhizosphere microbial communities [52, 53], and are frequently categorised as Plant Growth Promoting Rhizobacteria (PRPR) [54, 55]; B. subtilis has also been shown effective in controlling Meloidogyne species [56]. More generally, B. subtilis represents an important organism for many biotechnology applications, and is classified as GRAS (generally regarded as safe) by the FDA [57, 58]. It is increasingly well served by the development of synthetic biology tools [59], and can persist in soil for long periods through the production of spores [60]. We modified B. subtilis to secrete a number of PPN NLPs, and found that transformed B. subtilis cultures confer significant levels of protection on tomato cv. Moneymaker against both M. incognita and G. pallida infective juveniles (FIG. 4). This proof of concept demonstration employed a commercial B. subtilis strain and signal peptide sequence. It has however been reported that signal peptide identity can have a significant influence on the level of protein/peptide secreted by B. subtilis [61, 62]. We anticipate that signal peptide optimisation efforts could increase plant protection levels. Likewise, assessing other rhizobacteria strains may enhance efficacy. The secretion of uNLP nematicides could also be more targeted if driven by a plant root exudate-responsive promoter [63, 64, 65, 66].

We also utilised the soil-dwelling microalgae, C. reinhardtii as a novel synthesis and delivery platform. Like B. subtilis, C. reinhardtii benefits from an improving suite of synthetic biology tools [67]. C. reinhardtii cultures secreting selected PPN NLPs also provided significant levels of protection to tomato cv. Moneymaker when challenged by either M. incognita or G. pallida infective juveniles (FIG. 4).

The NLP screening approach employed here may underestimate the efficacy achievable through a continuous transgenic delivery (FIGS. 1, 2). For example, exogenous NLP-15b exposure inhibits G. pallida chemotaxis, but does not inhibit host invasion (FIG. 2). However, when NLP-15b is delivered continuously to G. pallida infective juveniles via microbial secretion, we observe a significant inhibition of tomato invasion relative to J2s exposed to unmodified B. subtilis (FIG. 4). This discrepency may be due to the recovery of G. pallida infective juveniles over the 24 hour timecourse of the tomato invasion assay. We expect that this may result in some false negative determinations in our NLP pre-screening approach.

Our data demonstrate that unamidated NLPs represent a new class of potent and specific plant protective nematicide which could be deployed as a transgenic trait in crop plants, or through soil microorganisms such as the B. subtilis and C. reinhardtii systems developed here. In particular, these non-crop delivery approaches could facilitate rapid deployment to many different crop plant species and cultivars. A key consideration in the development of PPN resistance traits must be the maintenance of genetic diversity across crop cultivars and isolates. This reduces the chance of widespread pathology from other pests as a result of genetic bottlenecks introduced by a single preferred transgenic cultivars.

### REFERENCES

1. Jones J T, Haegeman A, Danchin E G J, Gaur H S, Helder J, Jones M G K, Kikuchi T, Manzanilla-Lopez R, Palomares-Ruis J E, Wesemael W M L, Perry R N. Top 10 plant-parasitic nematodes in molecular plant pathology. Molecular Plant Pathology. 2013; 14(9): 946-961.

2. Nicol J M, Stirling G R, Turner S J, Coyne D L, de Nijs L, Hockland S, Maafi Z T. Current nematode threats to world agriculture. Genomics and Molecular Genetics of Plant-Nematode Interactions (Jones J T, Gheysen G, Fenoll C., eds). Heidelberg: Springer. 2011; pp. 21-44

3. Council of the European Union. 1991. Council Directive 91/414/EEC of 15 Jul. 1991 concerning the placing of plant protection products on the market. Official Journal. 2005; L 230: 1-32.

4. UNEP. Montreal protocol on substances that deplete the ozone layer. 2014 report of the methyl bromide technical options committee. 2015; p 13. ISBN: 978-9966-076-08-3.

5. Babich H, Davis D L, Stotzky G. Dibromochloropropane (DBCP): a review. The Science of Total Environment. 1981; 17: 207-221.

6. Lilley C J, Urwin P E, Johnston K A, and Atkinson H J. Preferential expression of a plant cystatin at nematode feeding sites confers resistance to Meloidogyne incognita and Globodera pallida. Plant Biotechnology Journal. 2004; 2(1): 3-12

7. Liu B, Hibbard J K, Urwin P E, Atkinson H J. The production of synthetic chemodisruptive peptides in planta disrupts the establishment of cyst nematodes. Plant Biotechnology Journal. 2005; 3(5): 487-96.

8. Green J, Wang D, Lilley C J, Urwin P E, Atkinson H J. Transgenic Potatoes for Potato Cyst Nematode Control Can Replace Pesticide Use without Impact on Soil Quality. Plos One. 2012; 7(2): e30973

9. Tripathi L, Babirye A, Roderick H, Tripathi J N, Changa C, Urwin P E, Tushemereirwe W K, Coyne D, Atkinson H J. Field resistance of transgenic plantain to nematodes has potential for future African food security. Nature, Scientific Reports 5, Article number: 8127. 2014.

10. Kyndt T, Ji H, Vanholme B, Gheysen G. Transcriptional silencing of RNAi constructs against nematode genes in Arabidopsis. NEMATOLOGY. 2013; 15(5):519-28.

11. Wang D, Jones L M, Urwin P E and Atkinson H J. A Synthetic Peptide Shows Retro- and Anterograde Neuronal Transport before Disrupting the Chemosensation of Plant-Pathogenic Nematodes. PLoS ONE. 2011; 6 (3): e17475

12. Maule A G, Geary T G, Bowman J W, Marks N J, Blair K L, Halton D W, Shaw C, Thompson D P. Inhibitory effects of nematode FMRFamide-related peptides (FaRPs) on muscle strips from Ascaris suum. Invertebrate Neuroscience. 1995; 1(3): 255-265.

13. Moffett C L, Beckett A M, Mousley A, Geary T G, Marks N J, Halton D W, Thompson D P, Maule A G. The ovijector of Ascaris suum: Multiple response types revealed by Caenorhabditis elegans FMRFamide-related peptides. International Journal of Parasitology. 2003; 33(8): 859-876.

14. McVeigh P, Geary T G, Marks N J, Maule A G. The FLP-side of nematodes. Trends in Parasitology. 2006; 22(8): 385-96.

15. Kimber M J, McKinney S, McMaster S, Day T A, Fleming C C, Maule A G. flp gene disruption in a parasitic nematode reveals motor dysfunction and unusual neuronal sensitivity to RNA interference. The Federation of American Societies for Experimental Biology Journal. 2007; 21(4):1233-43.

16. Li C, and Kim K. 2008. Neuropeptides. The C. elegans Research Community, WormBook.

17. McVeigh P, Alexander-Bowman S, Veal E, Mousley A, Marks N J, Maule A G. Neuropeptide-like protein diversity in phylum Nematoda. International Journal for Parasitology. 2008; 38(13): 1493-1503.

18. Marks N J, Maule A G. Neuropeptides in helminths: Occurrence and distribution. Advances in Experimental Medicine and Biology. 2010; 692: 49-77.

19. McVeigh P, Atkinson L, Marks N J, Mousley A, Dalzell J J, Sluder A, Hammerland L, Maule A G. Parasite neuropeptide biology: Seeding rational drug target selection? International Journal for Parasitology: Drugs and Drug Resistance. 2012; (2): 76-91.

20. Atkinson L E, Stevenson M, McCoy C J, Marks N J, Fleming C, Zamanian M, Day T A, Kimber M J, Maule A G, Mousley A. flp-32 Ligand/receptor silencing phenocopy faster plant pathogenic nematodes. PLoS Pathogens. 2013; 9(2):e1003169.

21. Peymen K, Watteyne J, Frooninckx L, Schoofs L, Beets I. The FMRFamide-Like Peptide Family in Nematodes. Frontiers in Endocrinology. 2014; 16; 5: 90.

22. Geary T G and Maule A G. Neuropeptide Systems as Targets for Parasite and Pest Control. Advances in Experimental Medicine and Biology. 2011; 692: v-vi.

23. Lee D J. The Biology of Nematodes. 2002.

24. Kimber M J, Fleming C C, Priord A, Jones J T, Halton D W, Maule A G. Localisation of Globodera pallida FMRFamide-related peptide encoding genes using in situ hybridisation. International Journal for Parasitology. 2002; 32(9): 1095-1105.

25. Fellowes R A, Maule A G, Marks N J, Geary T G, Thompson D P, Shaw C, Halton D W. Modulation of the motility of the vagina vera of Ascaris suum in vitro by FMRF amide-related peptides. Parasitology. 1998; 116: 277-287.

26. Bowman J W, Friedman A R, Thompson D P, Maule A G, Alexander-Bowman S J, and Geary T G. Structure-activity relationships of an inhibitory nematode FMRFamide-related peptide, SDPNFLRFamide (PF1), on Ascaris suum muscle. International Journal of Parasitology. 2002; 32: 1765-1771.

27. Kubiak T M, Larsen M J, Nulf S C, Zantello M R, Burton K J, Bowman J W, Modric T, and Lowery, D E. Differential activation of “social” and “solitary” variants of the Caenorhabditis elegans G protein-coupled receptor NPR-1 by its cognate ligand AF9. The Journal of Biological Chemistry. 2003; 278: 33724-33729.

28. Kubiak T M, Larsen M J, Zantello M R, Bowman J W, Nulf S C, and Lowery D E. Functional annotation of the putative orphan Caenorhabditis elegans G-protein-coupled receptor C1006.2 as a FLP15 peptide receptor. The Journal of Biological Chemistry. 2003; 278: 42115-42120.

29. Trailovic S M, Clark C L, Robertson A P, and Martin R J. Brief application of AF2 produces long lasting potentiation of nAChR responses in Ascaris suum. Molecular and Biochemical Parasitology. 2005; 139: 51-64.

30. Mertens I, Clinckspoor I, Janssen T, Nachman R, and Schoofs L. FMRFamide-related peptide ligands activate the Caenorhabditis elegans orphan GPCR Y59H11AL.1. Peptides. 2006; 27: 1291-1296

31. Mousley A, Maule A G, Halton D W, Marks N J. Inter-phyla studies on neuropeptides: the potential for broad-spectrum anthelmintic and/or endectocide discovery. Parasitology. 2005; 131: 143-67.

32. McVeigh P, Leech S, Mair G R, Marks N J, Geary T G, and Maule A G. Analysis of FMRFamide-like peptide (FLP) diversity in phylum Nematoda. International Journal of Parasitology. 2005; 35: 1043-1060.

33. Holden-Dye L, and Walker R J. Anthelmintic drugs. The C. elegans Research Community, WormBook. 2007.

34. James C E, Hudson A L, Daveyemail M W. Drug resistance mechanisms in helminths: is it survival of the fittest? Trends in Parasitology. 2009; 25(7), 328-335.

35. Kodama E, Kuhara A, Mohri-Shiomi A, Kimura K D, Okumura M, Tomioka M, lino Y, and Mori I. Insulin-like signaling and the neural circuit for integrative behavior in C. elegans. Genes and Development. 2006; 20: 2955-2960.

36. Duret L, Guex N, Peitsch M C, Bairoch A. New insulin-like proteins with atypical disulfide bond pattern characterized in Caenorhabditis elegans by comparative sequence analysis and homology modeling. Genome Research. 1998; 8: 348-353.

37. Gregoire F M, Chomiki N, Kachinskas D, Warden C H. Cloning and developmental regulation of a novel member of the insulin-like gene family in Caenorhabditis elegans. Biochemical and Biophysical Research Communications. 1998; 249(2): 385-390.

38. Smit A B, van Kesteren R E, Li KW, Van Minnen J, Spijker S, Van Heerikhuizen H, Geraerts W P. Towards understanding the role of insulin in the brain: Lessons from insulin-related signaling systems in the invertebrate brain. Progress in Neurobiology. 1998; 54:35-54.

39. Kawano T, Ito Y, Ishiguro M, Takuwa K, Nakajima T, Kimura Y. Molecular cloning and characterization of a new insulin/IGF-like peptide of the nematode Caenorhabditis elegans. Biochemical and Biophysical Research Communications. 2000; 273: 431-436.

40. Pierce, S B, Costa M, Wisotzkey R, Devadhar S, Homburger S A, Buchman A R, Ferguson K C, Heller J, Platt D M, Pasquinelli A A, Liu L X, Doberstein S K and Ruvkun G. Regulation of DAF-2 receptor signaling by human insulin and ins-1, a member of the unusually large and diverse C. elegans insulin gene family. Genes and Development. 2001; 15: 672-678.

41. Nathoo A N, Moeller R A, Westlund B A, and Hart A C. Identification of neuropeptide-like protein gene families in Caenorhabditis elegans and other species. Proceedings of the National Academy of Sciences. 2001; 98: 14000-14005.

42. Li W, Kennedy S G, and Ruvkun G. daf-28 encodes a C. elegans insulin superfamily member that is regulated by environmental cues and acts in the DAF-2 signaling pathway. Genes and Development. 2003; 17: 844-858.

43. Kim K, and Li C. Expression and regulation of an FMRFamide-related neuropeptide gene family in Caenorhabditis elegans. The Journal of Comparative Neurobiology. 2004; 475: 40-550.

44. Atkinson L E, Miskelly I R, Moffett C L, McCoy C J, Maule A G, Marks N J, Mousley A. Unraveling flp-11/flp-32 dichotomy in nematodes. International Journal of Parasitology. 2016; 46(11): 723-36.

45. Morris R, Wilson L, Warnock N D, Carrizo D, Cox D, Sturrock M, McGrath K, Maule A G, Dalzell J. A neuropeptide modulates sensory perception in the entomopathogenic nematode Steinernema carpocapsae. 2016; bioRxiv doi: http://dx.doi.orq/10.1101/061101

46. Abad P, Gouzy J, Aury J M, Castagnone-Sereno P, Danchin E G, Deleury E, Perfus-Barbeoch L, Anthouard V, Artiguenave F, Blok V C, Caillaud M C, Coutinho P M, Dasilva C, De Luca F, Deau F, Esquibet M, Flutre T, Goldstone J V, Hamamouch N, Hewezi T, Jaillon O, Jubin C, Leonetti P, Magliano M, Maier T R, Markov G V, McVeigh P, Pesole G, Poulain J, Robinson-Rechavi M, Sallet E, Ségurens B, Steinbach D, Tytgat T, Ugarte E, van Ghelder C, Veronico P, Baum T J, Blaxter M, Bleve-Zacheo T, Davis E L, Ewbank J J, Favery B, Grenier E, Henrissat B, Jones J T, Laudet V, Maule A G, Quesneville H, Rosso M N, Schiex T, Smant G, Weissenbach J, Wincker P. Genome sequence of the metazoan plant-parasitic nematode Meloidogyne incognita. Nature Biotechnology. 2008; 26(8): 909-15.

47. Cotton J A, Lilley C J, Jones L M, Kikuchi T, Reid A J, Thorpe P, Tsai I J, Beasley H, Blok V, Cock P J A, Akker S E, Holroyd N, Hunt M, Mantelin S, Naghra H, Pain A, Palomares-Rius J E, Zarowiecki M, Berriman M, Jones J T, and Urwin P E. The genome and life-stage specific transcriptomes of Globodera pallida elucidate key aspects of plant parasitism by a cyst nematode. Genome Biology. 2014; 15(3): R43.

48. Bargmann C I, Hartwieg E, and Horvitz H R. Odorant-selective genes and neurons mediate olfaction in C. elegans. Cell. 1993; 74: 515-527.

49. Bargmann C I. Chemosensation in C. elegans. The C. elegans Research Community, WormBook. 2006.

50. Maule A G, Shaw C, Bowman J W, Halton D W, Thompson D P, Thim L, Kubiak T M, Martin R A, Geary T G. Isolation and preliminary biological characterization of KPNFIRFamide, a novel FMRFamide-related peptide from the free-living nematode, Panagrellus redivivus. Peptides. 1995; 16(1): 87-93.

51. Davis R E, Stretton A O. Structure-activity relationships of 18 endogenous neuropeptides on the motor nervous system of the nematode Ascaris suum. Peptides. 2001 ; 22(1):7-23.

52. Hirooka K. Transcriptional response machineries of Bacillus subtilis conducive to plant growth promotion. Bioscience, Biotechnology, and Biochemistry. 2014; 78(9): 1471-1484.

53. Fall R, Kinsinger R F, Wheeler K A. A simple method to isolate biofilm-forming Bacillus subtilis and related species from plant roots. Systematic and Applied Microbiology. 2004; 27(3): 372-379.

54. Compant S, Duffy B, Nowak J, Clement C, Barka E A. Use of plant growth-promoting bacteria for biocontrol of plant diseases: principles, mechanisms of action, and future prospects. Applied and Environmental Microbiology 71(9): 4951-4959.

55. Vessey J K. Plant growth promoting rhizobacteria as biofertilizers. Plant and Soil. 2003; 255(2): 571-586.

56. Wepuhkhulu M, Kimenju J, Anyango B, Wachira P and Kyallo G. Effect of soil fertility management practices and Bacillus subtilis on plant parasitic nematodes associated with common bean, Phaseolus vulgaris. Tropical and Subtropical Agroecosystems. 2011; 13: 27-34.

57. Van Dijl J M, Hecker M. Bacillus subtilis: from soil bacterium to super-secreting cell factory. Microbial Cell Factories. 2013; 12:3.

58. Das K, Mukherjee A K. Crude petroleum-oil biodegradation efficiency of Bacillus subtilis and Pseudomonas aeruginosa strains isolated from a petroleum-oil contaminated soil from North-East India. Bioresource Technology. 2004; 98(7): 1339-1345.

59. Guiziou S, Sauveplane V, Chang H J, Clerté C, Declerck N, Jules M, Bonnet J. A part toolbox to tune genetic expression in Bacillus subtilis. Nucleic Acids Research. 2016; 44(15): 7495-508.

60. Wood J P, Meyer K M, Kelly T J, Choi Y W, Rogers J V, Riggs K B, and Willenberg Z J. Environmental Persistence of Bacillus anthracis and Bacillus subtilis Spores. PLoS One 2015; 10(9): e0138083.

61. Brockmeier U, Caspers M, Freudl R, Jockwer A, Noll T, Eggert T. Systematic screening of all signal peptides from Bacillus subtilis: a powerful strategy in optimizing heterologous protein secretion in Gram-positive bacteria. Journal of Molecular Biology. 2006; 362: 393-402.

62. Degering C, Eggert T, Puls M, Bongaerts J, Evers S, Maurer K H, Jaeger K H. Optimization of protease secretion in Bacillus subtilis and Bacillus licheniformis by screening of homologous and heterologous signal peptides. Applied and Environmental Microbiology. 2010; 76(19): 6370-6376.

63. Dunn A K, Klimowicz A K, and Handelsman J. Use of a Promoter Trap To Identify Bacillus cereus Genes Regulated by Tomato Seed Exudate and a Rhizosphere Resident, Pseudomonas aureofaciens. Applied and Environmental Microbiology. 2003; 69(2): 1197-1205.

64. Xie S, Wu H, Chen L, Zang H, Xie Y, and Gao X. Transcriptome profiling of Bacillus subtilis OKB105 in response to rice seedlings. BMC Microbiology. 2015; 15(1): 21.

65. Zhang N, Yang D, Wang D, Miao Y, Shao J, Zhou X, Xu Z, Li Q, Feng H, Li S, Shen Q, and Zhang R. Whole transcriptomic analysis of the plant-beneficial rhizobacteriurn Bacillus amyloliquefaciens SQR9 during enhanced biofilm formation regulated by maize root exudates. BMC Genomics. 2015; 16(1): 685.

66. Xie S, Wu H, Chen L, Zang H, Xie y, and Gao X. Transcriptome profiling of Bacillus subtilis OKB105 in response to rice seedlings. BMC Microbiology. 2015; 15(1): 21.

67. Merchant S S, Prochnik S E, Vallon O, Harris E H, Karpowicz S J, Witman G B, Terry A, Salamov A, Fritz-Laylin L K, Maréchal-Drouard L, Marshall W F, Qu L H, Nelson D R, Sanderfoot A A, Spalding M H, Kapitonov V V, Ren Q, Ferris P, Lindquist E, Shapiro H, Lucas S M, Grimwood J, Schmutz J, Cardol P, Cerutti H, Chanfreau G, Chen C L, Cognat V, Croft M T, Dent R, Dutcher S, Fernández E, Fukuzawa H, González-Ballester D, González-Halphen D, Hallmann A, Hanikenne M, Hippler M, Inwood W, Jabbari K, Kalanon M, Kuras R, Lefebvre P A, Lemaire S D, Lobanov A V, Lohr M, Manuell A, Meier I, Mets L, Mittag M, Mittelmeier T, Moroney J V, Moseley J, Napoli C, Nedelcu A M, Niyogi K, Novoselov S V, Paulsen I T, Pazour G, Purton S, Ral J P, Riaño-Pachón D M, Riekhof W, Rymarquis L, Schroda M, Stern D, Umen J, Willows R, Wilson N, Zimmer S L, Allmer J, Balk J, Bisova K, Chen C J, Elias M, Gendler K, Hauser C, Lamb M R, Ledford H, Long J C, Minagawa J, Page M D, Pan J, Pootakham W, Roje S, Rose A, Stahlberg E, Terauchi A M, Yang P, Ball S, Bowler C, Dieckmann C L, Gladyshev V N, Green P, Jorgensen R, Mayfield S, Mueller-Roeber B, Rajamani S, Sayre R T, Brokstein P, Dubchak I, Goodstein D, Hornick L, Huang Y W, Jhaveri J, Luo Y, Martinez D, Ngau W C, Otillar B, Poliakov A, Porter A, Szajkowski L, Werner G, Zhou K, Grigoriev I V, Rokhsar D S, Grossman A R. The Chlamydomonas genome reveals the evolution of key animal and plant functions. Science. 2007; 318(5848): 245-50.

68. Hart A C. Behavior. The C. elegans Research Community, WormBook. 2006.

69. Bybd, D. W, J R., T. Kirkpatrick, T. J R, and K. R. Barker, K.R. An Improved Technique for Clearing and Staining Plant Tissues for Detection of Nematodes. Journal of Nematology. 1983; 15(1): 142-143.

70. Stiernagle, T. Maintenance of C. elegans. The C. elegans Research Community, WormBook. 2006.

71. White G. A method for obtaining infective nematode larvae from cultures. American Association for the Advancement of Science. 1927; 66 (1709): 302-3.

The invention is not limited to the embodiments described herein but can be amended or modified without departing from the scope of the present invention.

1. A peptide comprising:
AA₁-AA₂-AA₃-F-D-AA₆-AA₇-AA₈-AA₉-AA₁₀-AA₁₁-AA₁₂-AA₁₃-AA₁₄-AA₁₅-AA₁₆-AA₁₇;
wherein AA₁ is selected from S, N and A;
wherein AA₂ is selected from S and A;
wherein AA₃ is selected from S, N and A;
wherein AA₆ is selected from S, L, D and A;
wherein AA₇ is selected from F, S and L;
wherein AA₈ is selected from V, T, M, A, F and G;
wherein AA₉ is selected from G, V and T;
wherein AA₁₀ is selected from R, K, P, S, G and N;
wherein AA₁₁ is selected from G and R;
wherein AA₁₂ is selected from F and G;
wherein AA₁₃ is selected from T and F;
wherein AA₁₄ is selected from G and T;
wherein AA₁₅ is selected from M, L, G and F;
wherein AA₁₆ is selected from D and M; and
wherein AA₁₇ is present or absent and, if present, is selected from T and D.

2. The peptide of claim 1, wherein the peptide comprises:
AA₁-AA₂-AA₃-F-D-AA₆-AA₇-AA₈-AA₉-AA₁₀-G-F-T-G-AA₁₅-D-AA_{17;}
wherein AA₁ is present or absent and, if present, is selected from S or A;
wherein AA₂ is present or absent and, if present, is S and A;
wherein AA₃ is selected from A, S and N;
wherein AA₆ is selected from S, L and A;
wherein AA₇ is selected from F and L;
wherein AA₈ is selected from V, T, M, A and G;
wherein AA₉ is selected from G and T;
wherein AA₁₀ is selected from R, K, P, S and N;
wherein AA₁₅ is selected from M, L and F; and
wherein AA₁₇ is present or absent and, if present, is selected from D and T.

3. The peptide of claim 1, wherein the peptide comprises:
AA₁-AA₂-AA₃-F-D-AA₆-AA₇-AA₈-AA₉-AA₁₀-G-F-T-G-AA₁₅-D-AA₁₇;
wherein AA₁ is absent;
wherein AA₂ is present or absent and, if present, is selected from S and A;
wherein AA₃ is selected from A and S;
wherein AA₆ is selected from S, L and A;
wherein AA₇ is selected from F and L;
wherein AA₈ is selected from V, T, M, A and G;
wherein AA₉ is selected from G and T;
wherein AA₁₀ is selected from R, K, P, S and N;
wherein AA₁₅ is selected from M, L and F; and
wherein AA₁₇ is present or absent and, if present, is T.

4. The peptide of claim 1, wherein the peptide comprises:
Gp-NLP-15a
SFDSLTGPGFTGLDT
Gp-NLP-15b
SFDSFTGPGFTGLD
Gp-NLP-15c
SFDSFTGSGFTGLD
Gp-NLP-15f
SFDSFMGPGFTGMD
Gp-NLP-15h
AFDLFTGPGFTGMD
Gp-NLP-15g
AFDSFTGPGFTGMD
Mi-NLP-15a
AFDSFGTPGFTGFD
Mi-NLP-15b
SFDSFTGPGFTGLD
Mi-NLP-15c
SFDSFVGKGFTGMD
Mi-NLP-15d
AFDSFGTPGFTGFD
Mi-NLP-15e
SAFDSFVGRGFTGMD
Mi-NLP-15f
AFDSFAGNGFTGFD
Mi-NLP-15g
NFDAFMGPGFTGLD
or
Mi-NLP-15h
AAFDSFVGRGFTGMD,

5. The peptide of claim 1, wherein the peptide comprises:
Mi-NLP-15b
SFDSFTGPGFTGLD
or
Mi-NLP-15e
SAFDSFVGRGFTGMD,

6-14. (canceled)

15. A nematicidal composition comprising a peptide as claimed in claim 1, or a mixture thereof, and a suitable carrier.

16. An expression vector comprising a peptide as claimed in claim 1.

17. The vector of claim 16, wherein a promoter is operably linked to the peptide.

18. A transgenic microorganism for expression of a peptide, the microorganism comprising an expression vector comprising a peptide as claimed in claim 1.

19. A method of treating plant parasitic nematodes, the method comprising providing a peptide as claimed in claim 1 on or adjacent the plant parasitic nematodes.

20. The peptide of claim 1, wherein the peptide comprises:
15b
SFDSFTGPGFTGLD
15e
SAFDSFVGRGFTGMD
9f
GGGRYFIRPFADQ
18a
FAPRQFAFA
14c
ALDMMEGDDFIGL
8d
AFDRLDNSFMLL
or a mixture thereof.

21. The peptide of claim 1, wherein the peptide comprises:
15b
SFDSFTGPGFTGLD.

22. A transgenic microorganism for expression of a peptide, the microorganism comprising an expression vector comprising a peptide as claimed in claim 1, wherein a promoter is operably linked to the peptide.

23. The peptide of claim 1, wherein the peptide consists of:
AA₁-AA₂-AA₃-F-D-AA₆-AA₇-AA₈-AA₉-AA₁₀-AA₁₁-AA₁₂-AA₁₃-AA₁₄-AA₁₅-AA₁₆-AA_{17;}
wherein AA₁ is selected from S, N and A;
wherein AA₂ is selected from S and A;
wherein AA₃ is selected from S, N and A;
wherein AA₆ is selected from S, L, D and A;
wherein AA₇ is selected from F, S and L;
wherein AA₈ is selected from V, T, M, A, F and G;
wherein AA₉ is selected from G, V and T;
wherein AA₁₀ is selected from R, K, P, S, G and N;
wherein AA₁₁ is selected from G and R;
wherein AA₁₂ is selected from F and G;
wherein AA₁₃ is selected from T and F;
wherein AA₁₄ is selected from G and T;
wherein AA₁₅ is selected from M, L, G and F;
wherein AA₁₆ is selected from D and M; and
wherein AA₁₇ is present or absent and, if present, is selected from T and D.

24. The peptide of claim 1, wherein the peptide consists of:
AA₁-AA₂-AA₃-F-D-AA₆-AA₇-AA₈-AA₉-AA₁₀-G-F-T-G-AA₁₅-D-AA₁₇;
wherein AA₁ is present or absent and, if present, is selected from S or A;
wherein AA₂ is present or absent and, if present, is S and A;
wherein AA₃ is selected from A, S and N;
wherein AA₆ is selected from S, L and A;
wherein AA₇ is selected from F and L;
wherein AA₈ is selected from V, T, M, A and G;
wherein AA₉ is selected from G and T;
wherein AA₁₀ is selected from R, K, P, S and N;
wherein AA₁₅ is selected from M, L and F; and
wherein AA₁₇ is present or absent and, if present, is selected from D and T.

25. The peptide of claim 1, wherein the peptide consists of:
AA₁-AA₂-AA₃-F-D-AA₆-AA₇-AA₈-AA₉-AA₁₀-G-F-T-G-AA₁₅-D-AA₁₇;
wherein AA₁ is absent;
wherein AA₂ is present or absent and, if present, is selected from S and A;
wherein AA₃ is selected from A and S;
wherein AA₆ is selected from S, L and A;
wherein AA₇ is selected from F and L;
wherein AA₈ is selected from V, T, M, A and G;
wherein AA₉ is selected from G and T;
wherein AA₁₀ is selected from R, K, P, S and N;
wherein AA₁₅ is selected from M, L and F; and
wherein AA₁₇ is present or absent and, if present, is T.

26. The peptide of claim 1, wherein the peptide consists of:
Gp-NLP-15a
SFDSLTGPGFTGLDT
Gp-NLP-15b
SFDSFTGPGFTGLD
Gp-NLP-15c
SFDSFTGSGFTGLD
Gp-NLP-15f
SFDSFMGPGFTGMD
Gp-NLP-15h
AFDLFTGPGFTGMD
Gp-NLP-15g
AFDSFTGPGFTGMD
Mi-NLP-15a
AFDSFGTPGFTGFD
Mi-NLP-15b
SFDSFTGPGFTGLD
Mi-NLP-15c
SFDSFVGKGFTGMD
Mi-NLP-15d
AFDSFGTPGFTGFD
Mi-NLP-15e
SAFDSFVGRGFTGMD
Mi-NLP-15f
AFDSFAGNGFTGFD
Mi-NLP-15g
NFDAFMGPGFTGLD
or
Mi-NLP-15h
AAFDSFVGRGFTGMD,

27. The peptide of claim 1, wherein the peptide consists of:
Mi-NLP-15b
SFDSFTGPGFTGLD
or
Mi-NLP-15e
SAFDSFVGRGFTGMD,

28. The peptide of claim 1, wherein the peptide consists of:
15b
SFDSFTGPGFTGLD
15e
SAFDSFVGRGFTGMD
9f
GGGRYFIRPFADQ
18a
FAPRQFAFA
14c
ALDMMEGDDFIGL
8d
AFDRLDNSFMLL
or a mixture thereof.

29. The peptide of claim 1, wherein the peptide consists of:
15b
SFDSFTGPGFTGLD.


SEQUENCE LISTING (53 sequences)

SEQ ID NO: 1 - protein, 25 aa, Globodera pallida
FSDDELAAMPLNDLYLSSPYAFGPF

SEQ ID NO: 2 - protein, 13 aa, Globodera pallida
SFDRLEESAFFGQ

SEQ ID NO: 3 - protein, 13 aa, Globodera pallida
ALDILESDDFGGF

SEQ ID NO: 4 - protein, 14 aa, Globodera pallida
ALDVMDGDGFGSFE

SEQ ID NO: 5 - protein, 13 aa, Globodera pallida
ALDTLEGDDFMGL

SEQ ID NO: 6 - protein, 13 aa, Globodera pallida
LNELEGDGFMGLD

SEQ ID NO: 7 - protein, 14 aa, Globodera pallida
ALDILDGDDFTGFS

SEQ ID NO: 8 - protein, 12 aa, Globodera pallida
ALDALEGNSFGF

SEQ ID NO: 9 - protein, 15 aa, Globodera pallida
SFDSLTGPGFTGLDT

SEQ ID NO: 10 - protein, 14 aa, Globodera pallida
SFDSFTGPGFTGLD

SEQ ID NO: 11 - protein, 14 aa, Globodera pallida
SFDSFTGSGFTGLD

SEQ ID NO: 12 - protein, 11 aa, Globodera pallida
AAFDTDFTNYD

SEQ ID NO: 13 - protein, 13 aa, Globodera pallida
FEPFDGYGFNGFE

SEQ ID NO: 14 - protein, 14 aa, Globodera pallida
SFDSFMGPGFTGMD

SEQ ID NO: 15 - protein, 14 aa, Globodera pallida
AFDSFTGPGFTGMD

SEQ ID NO: 16 - protein, 14 aa, Globodera pallida
AFDLFTGPGFTGMD

SEQ ID NO: 17 - protein, 14 aa, Globodera pallida
GGARAFNFFAPPDE

SEQ ID NO: 18 - protein, 13 aa, Globodera pallida
GGARAFNFFAPDE

SEQ ID NO: 19 - protein, 19 aa, Globodera pallida
GGTRAFNFFVSDALPSSYE

SEQ ID NO: 20 - protein, 18 aa, Globodera pallida
SGIQTFRDDYDEKQAGEL

SEQ ID NO: 21 - protein, 21 aa, Globodera pallida
AGGRLFRMVDLPDGDDFVPEG

SEQ ID NO: 22 - protein, 16 aa, Globodera pallida
GGARPFYGGGYMDGTW

SEQ ID NO: 23 - protein, 20 aa, Globodera pallida
AGGRYFMRHFDDSPFAGWMA

SEQ ID NO: 24 - protein, 22 aa, Globodera pallida
GGARAFFGDADGPFNSASYWAP

SEQ ID NO: 25 - protein, 20 aa, Globodera pallida
GGARAFNGAEETLLNVANLA

SEQ ID NO: 26 - protein, 13 aa, Meloidogyne incognita
SSLASGRIGFRPA

SEQ ID NO: 27 - protein, 16 aa, Meloidogyne incognita
AFDRLDVSPFDFDAMT

SEQ ID NO: 28 - protein, 22 aa, Meloidogyne incognita
FNDDELSSLPFNFEYFPSLDTH

SEQ ID NO: 29 - protein, 13 aa, Meloidogyne incognita
AFDRLEDSGFFGL

SEQ ID NO: 30 - protein, 12 aa, Meloidogyne incognita
AFDRLDNSFMLL

SEQ ID NO: 31 - protein, 18 aa, Meloidogyne incognita
AGARAFQRPDFDDASYEL

SEQ ID NO: 32 - protein, 10 aa, Meloidogyne incognita
GGARTFLVGE

SEQ ID NO: 33 - protein, 11 aa, Meloidogyne incognita
GGARAFAKLEE

SEQ ID NO: 34 - protein, 9 aa, Meloidogyne incognita
GGARPFYEE

SEQ ID NO: 35 - protein, 17 aa, Meloidogyne incognita
GGARPFYGFFGGGEGTW

SEQ ID NO: 36 - protein, 13 aa, Meloidogyne incognita
GGGRYFIRPFADQ

SEQ ID NO: 37 - protein, 14 aa, Meloidogyne incognita
ALDMLEGDDFIGMQ

SEQ ID NO: 38 - protein, 15 aa, Meloidogyne incognita
ALDLMEGDGFGGGFD

SEQ ID NO: 39 - protein, 13 aa, Meloidogyne incognita
ALDMMEGDDFIGL

SEQ ID NO: 40 - protein, 14 aa, Meloidogyne incognita
AFDSFGTPGFTGFD

SEQ ID NO: 41 - protein, 14 aa, Meloidogyne incognita
SFDSFTGPGFTGLD

SEQ ID NO: 42 - protein, 14 aa, Meloidogyne incognita
SFDSFVGKGFTGMD

SEQ ID NO: 43 - protein, 14 aa, Meloidogyne incognita
AFDSFGTPGFTGFD

SEQ ID NO: 44 - protein, 15 aa, Meloidogyne incognita
SAFDSFVGRGFTGMD

SEQ ID NO: 45 - protein, 14 aa, Meloidogyne incognita
AFDSFAGNGFTGFD

SEQ ID NO: 46 - protein, 14 aa, Meloidogyne incognita
NFDAFMGPGFTGLD

SEQ ID NO: 47 - protein, 15 aa, Meloidogyne incognita
AAFDSFVGRGFTGMD

SEQ ID NO: 48 - protein, 9 aa, Meloidogyne incognita
FAPRQFAFA

SEQ ID NO: 49 - protein, 8 aa, Meloidogyne incognita
GMRNFAFA

SEQ ID NO: 50 - protein, 15 aa, Meloidogyne incognita
SFGDYPFGSRTFAFA

SEQ ID NO: 51 - protein, 12 aa, Meloidogyne incognita
AAENFDENNDIN

SEQ ID NO: 52 - protein, 16 aa, Meloidogyne incognita
SSQFGGENSFARFAFA

SEQ ID NO: 53 - protein, 7 aa, Meloidogyne incognita
MVSWQPV